(19) United States
(12) Patent Application Publication
(10) Pub. No.: US 2021/0145609 A1
(43) Pub. Date: May 20, 2021

(54) NIPPLE COVER

(71) Applicant: BITTY LOVE LLC, Los Angeles, CA (US)

(72) Inventor: Sandy Novak, Los Angeles, CA (US)

(21) Appl. No.: 16/685,945

(22) Filed: Nov. 15, 2019

(51) Int. Cl. A61F 2/52; A41C 3/12

(52) U.S. Cl. CPC A61F 2/52; A61F 2002/7806; A61F 2002/526; A41C 3/128

(57) ABSTRACT

A nipple cover comprising: an adhesive portion and a non-adhesive portion. The non-adhesive portion is placed near the center of the adhesive nipple cover, in order to create a combined nipple cover with a non-adhesive zone near the center of the adhesive face of the nipple cover. The resulting nipple cover may be placed directly onto a wearer's body to comfortably provide protection and reduced nipple visibility to outside observers.

### FIELD OF USE

The present disclosure relates generally to nipple covers that are worn to minimize the visible appearance of the wearer's nipples as seen through the wearer's clothing. More specifically, the present disclosure relates to an adhesive nipple cover that sticks directly to the skin of the wearer, which is used in connection with a smaller, non-adhesive portion. The non-adhesive portion is placed directly over and in contact with the nipple/areola area of the wearer in order to maximize comfort over prolonged periods of wear.

### BACKGROUND

Various types of nipple covers currently exist, which serve to mask the visible appearance of the wearer's nipples to an outside observer. While certain clothing options, such as various types of bras, may help to limit the visibility of a person's nipples to outside observers, there may be situations in which additional coverage or layering is desired. This may, for example, be the case when wearing thin clothing or a bra with no pad or is thinly-padded. There may also be situations in which other common nipple-covering options (such as a bra) are not desirable, particularly if backless clothing is worn that would expose the wearer's bra strap.

Nipple covers are a way to address a person's nipple visibility concerns, even when wearing a non-padded bra or no bra at all, thin clothing, or backless clothing (such as a dress) in which a bra strap would be visible. Nipple covers prior to the present disclosure are often adhesive based that are applied directly to the wearer's breast area in order to help provide a more uniform and smoothed out appearance of the breast and nipple area. This smoothed out appearance may reduce undesirable nipple visibility.

However, the adhesives used by some nipple covers may make users uncomfortable when wearing them for prolonged periods of time. This is partly due to the fact that nipples tend to be very sensitive, such that the adhesive used by many nipple covers irritates the wearer's nipples the longer the cover is worn. Additionally, some nipple covers may lose adhesion as the wearer sweats while wearing the nipple cover. This issue of sweating and lack of adhesion is particularly pronounced on the wearer's nipple, in comparison to the surrounding areola and larger skin area.

Therefore, what is needed is a nipple cover which provides maximum comfort for a wearer, particularly at the point of contact between the wearer's nipple and the nipple cover even when worn for extended periods of time, and which will remain adhered to the wearer even as the wearer sweats.

### SUMMARY

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

In accordance with the embodiments disclosed herein, the present disclosure is directed to an adhesive nipple cover and a non-adhesive portion. The adhesive nipple cover adheres directly to the wearer's skin, while the non-adhesive portion adheres to the adhesive nipple cover but not directly to the wearer's skin.

The adhesive nipple cover may be worn with or without the non-adhesive portion. The adhesive nipple cover preferably comprises an adhesive side, allowing easy and direct application to the wearer's skin, and a non-adhesive side, in order to avoid sticking to the wearer's clothes. The adhesive nipple cover may range in size, from approximately 2-4 inches in diameter.

Another feature of the adhesive nipple cover includes the ability to be washed and reused after removal, allowing for repeated use.

In some embodiments, the adhesive nipple cover may be made of a thin, flexible, and/or elastic material that allows the adhesive nipple cover to conform closely to the body shape of the wearer. In other embodiments, the color of the adhesive nipple cover may vary in order to match the skin tone of the wearer or the clothing color of the wearer.

In other embodiments, the adhesive nipple cover may further comprise a non-adhesive portion. It is contemplated that the non-adhesive portion be a smaller size than the adhesive nipple cover, such that the non-adhesive portion may be placed on the center of the adhesive side of the adhesive nipple cover. Placing the non-adhesive portion onto the adhesive side of the adhesive nipple cover creates a non-adhesive zone in the center of the adhesive side of the adhesive nipple cover. In some embodiments, the adhesive may preferably be polysiloxane based.

The combined nipple cover (comprising the adhesive nipple cover and the non-adhesive portion) may then be placed onto the wearer's breast, preferably by placing the non-adhesive zone of the combined nipple cover directly over the wearer's nipple. The remaining adhesive zone of the combined nipple cover may provide sufficient adhesion to comfortably maintain the combined nipple cover on the wearer's body for prolonged periods of time.

The non-adhesive zone over the wearer's nipple provides for a more comfortable nipple cover experience, by protecting the wearer's nipple from irritation by contact with the adhesive zone of the combined nipple cover. Additionally, the presence of the non-adhesive portion near the center of the combined nipple cover further smooths out the breast and nipple area of the wearer to outside observers.

In some embodiments, the non-adhesive portion may range in size from approximately ¾″ to ½″ in diameter. The non-adhesive portion is preferably made of a similar flexible, rubbery material as the adhesive nipple cover, allowing the non-adhesive portion to bend or flex to conform to the shape of the wearer's body.

One embodiment may be a nipple cover, comprising: an interior; an outer surface; and an inner surface, wherein the inner surface comprises an adhesive portion; wherein the interior is between the outer surface and the inner surface; wherein the outer surface is convex and the inner surface is concave, such that the nipple cover is curved and wherein the inner surface is configured to matingly engage with a wearer's breast, such that the wearer's nipple is covered by the nipple cover. Preferably, the inner surface further comprises a non-adhesive portion, which may be at a center of the inner surface, such that the non-adhesive portion is surrounded by the adhesive portion. The non-adhesive portion may be separable from the nipple cover or may be permanently attached or may be unitary with the nipple cover. The nipple cover may preferably be made from a flexible and soft material. In various embodiments, the nipple cover maybe made from one or more of the materials selected from the group of materials consisting of: natural rubber; synthetic rubber; silicone; silicone rubber; neoprene; foam rubber; nitrile; latex; and/or combinations thereof.

Another embodiment may be a nipple cover, comprising: an interior; an outer surface; and an inner surface, wherein the inner surface comprises an adhesive which entirely covers the inner surface; and a non-adhesive portion; wherein the interior is between the outer surface and the inner surface; wherein the inner surface and the non-adhesive portion are configured to matingly engage, such that the non-adhesive portion is positioned at a center of the inner surface; wherein the inner surface is concave; and wherein the inner surface and the non-adhesive portion are configured to matingly engage with a wearer's breast, such that a nipple of the wearer is covered by the nipple cover. The non-adhesive portion may directly cover the nipple, such that the adhesive does not engage directly with the nipple. The interior, the outer surface, the inner surface, and the non-adhesive portion are made from one or more of the materials selected from the group of materials consisting of: natural rubber; synthetic rubber; silicone; silicone rubber; neoprene; foam rubber; nitrile; latex; and combinations thereof.

Another embodiment may be a nipple cover, comprising: an outer surface; an inner surface, wherein the inner surface comprises an adhesive which entirely covers the inner surface; and a non-adhesive portion; wherein the inner surface and the non-adhesive portion are configured to matingly engage, such that the non-adhesive portion is positioned at a center of the inner surface; wherein the inner surface is concave; wherein the inner surface and the non-adhesive portion are configured to matingly engage with a wearer's breast, such that a nipple of the wearer is covered by the nipple cover; and wherein the non-adhesive portion directly covers the nipple, such that the adhesive does not engage directly with the nipple. The interior, the outer surface, the inner surface, and the non-adhesive portion may be made from one or more of the materials selected from the group of materials consisting of: natural rubber; synthetic rubber; silicone; silicone rubber; neoprene; foam rubber; nitrile; latex; and combinations thereof.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an illustration of an outer perspective view of one embodiment of a nipple cover.

FIG. 2 is an illustration of an inner view of one embodiment of a nipple cover showing the inner adhesive cover.

FIG. 3 is an illustration of an outer view of one embodiment of a nipple cover and a non-adhesive portion.

FIG. 4 is an illustration of an inner view of one embodiment of a nipple cover and an outer view of a non-adhesive portion.

FIG. 5 is an illustration of an inner view of one embodiment of a nipple cover with the non-adhesive portion in place.

FIG. 6 is an illustration of an inner perspective view of one embodiment of a nipple cover.

FIG. 7 is an illustration of a cross-section view of one embodiment of the nipple cover of FIG. 5.

### DETAILED DESCRIPTION OF THE ILLUSTRATIVE EMBODIMENTS

Before the present methods and systems are disclosed and described, it is to be understood that the methods and systems are not limited to specific methods, specific components, or to particular implementations. The methods and systems disclosed and described herein may be understood more readily by reference to the following detailed description of preferred embodiments and the examples included therein and to the Figures and their previous and following description. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where the event or circumstance occurs and instances where it does not.

The term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item or result.

As used herein, the terms “approximately” and “about” generally refer to a deviance of within 5% of the indicated number or range of numbers. In one embodiment, the term “approximately” and “about” may refer to a deviance of between 0.001-10% from the indicated number or range of numbers. Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

As used herein, the term “nipple cover” refers generally to a device that is worn on a person's body in order to smooth out the breast and nipple area to minimize the visibility of the person's nipples when viewed by an outside observer.

As used herein, the term “non-adhesive portion” refers to that portion of the device of the present disclosure that does not stick to the user's skin and is worn in connection with a nipple cover on a wearer's body in order to provide additional protection and comfort specifically for the wearer's nipple.

As used herein, the term “combined nipple cover” refers to any combination of a nipple cover with a non-adhesive portion as described in the present disclosure.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details.

In accordance with the embodiments disclosed herein, the present disclosure is directed to a nipple cover which may be easily and readily applied to a wearer's body in order to reduce the visibility of the wearer's nipples when observed by others. The nipple cover of the present disclosure serves as a convenient and comfortable way to reduce nipple visibility by masking or smoothing out the wearer's breast and nipple area.

Before the present methods and systems are disclosed and described in detail, it is to be understood that the methods and systems are not limited to specific methods, specific components, or to particular implementations. It is also to be understood that the terminology used herein is for the purpose of describing particular embodiments only and is not intended to be limiting.

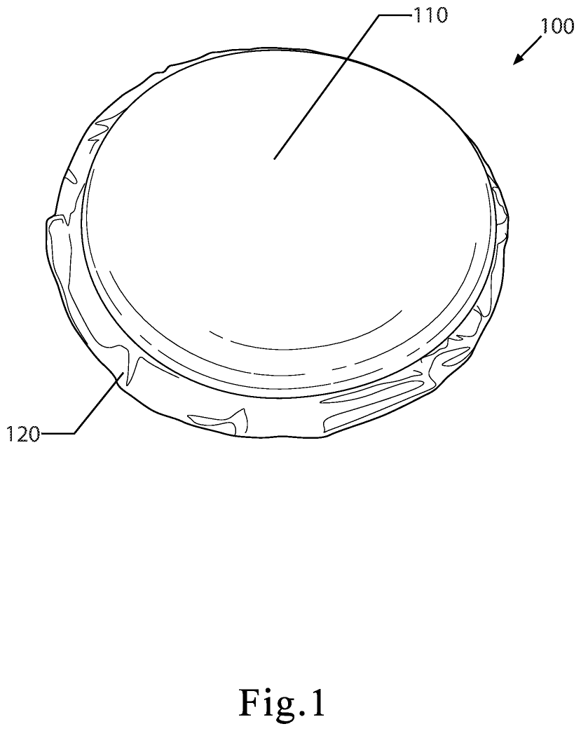

FIG. 1 is an illustration of an outer perspective view of one embodiment of a nipple cover. As shown in FIG. 1, the nipple cover 100 may have an outer surface 110. The inner surface (not shown in FIG. 1) may have an adhesive impregnated on it. Accordingly, an inner adhesive cover 120 may be used to keep the adhesive clean before use. FIG. 1 shows that the nipple cover 100 may have a substantially convex outer surface 110

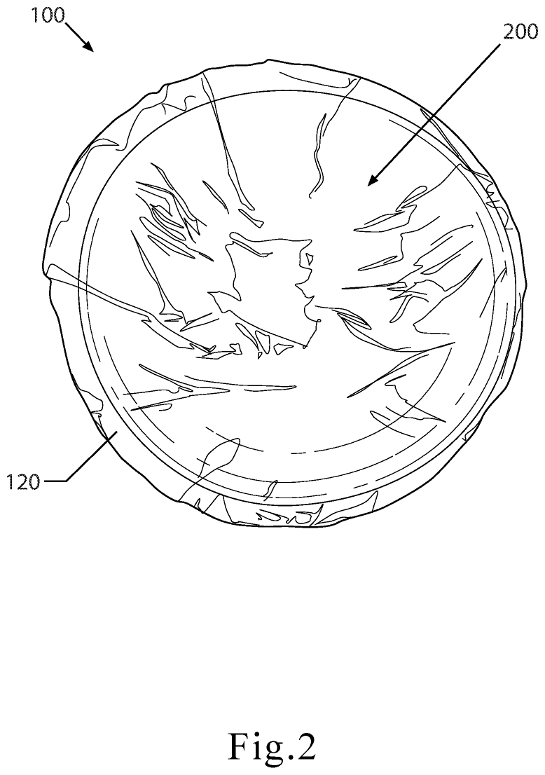

FIG. 2 is an illustration of an inner view of one embodiment of a nipple cover showing the inner adhesive cover. FIG. 2 shows that the nipple cover 100 may have an inner surface 200 that may be covered by inner adhesive cover 120 to keep the adhesive clean, fresh, and/or sterile. FIG. 2 shows that the inner surface 200 may be concave. The inner surface 200 is designed to fit over a wearer's nipple so that the nipple is substantially covered, typically for modesty purposes. The nipple cover 100 may be a range of colors to blend with the wearer's skin, or it can be colored to blend with what clothing is being worn. The nipple cover 100 is shown as being substantially round and hemispherical, but can be of any shape that may partially, substantially, or entirely cover the user's nipple.

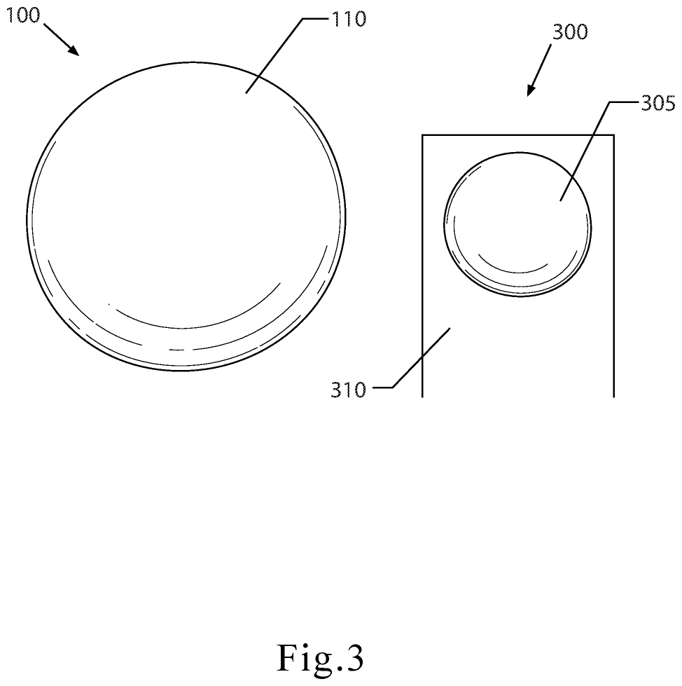

FIG. 3 is an illustration of an outer view of one embodiment of a nipple cover and a non-adhesive portion. As shown in FIG. 3, the nipple cover 100 may comprise an outer surface 110 and may be combined with non-adhesive portion 300. Non-adhesive portion 300 may have an outer surface 305. The non-adhesive portion may be mounted on backing 310, which may keep the non-adhesive portion secure during transportation and may keep an inner surface clean.

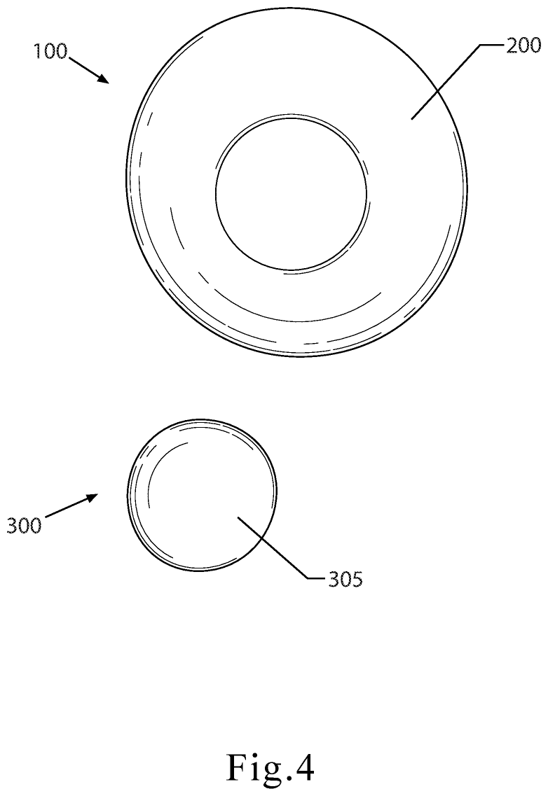

FIG. 4 is an illustration of an inner view of one embodiment of a nipple cover and an outer view of a non-adhesive portion. FIG. 4 shows that the inner surface 200 of nipple cover 100 has been uncovered and the non-adhesive portion 300 has been removed from the backing. FIG. 4 shows how the inner surface 200 may be effectively and substantially covered with an adhesive. In other embodiments, the adhesive may be purposefully left off certain portions, such as the middle, to create a non-adhesive portion. FIG. 4 shows how the outer surface 305 of the non-adhesive portion 300 may be configured to matingly engage with a center portion of the inner surface 200 of nipple cover 100.

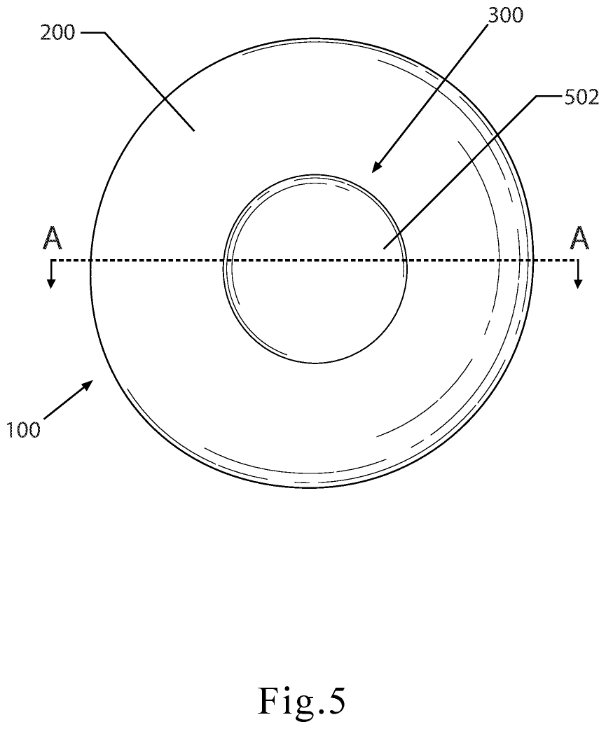

FIG. 5 is an illustration of an inner view of one embodiment of a nipple cover with the non-adhesive portion in place. FIG. 5 shows that the non-adhesive portion 300 has been matingly engaged with the inner surface 200 of the nipple cover 100. The inner surface 502 of the non-adhesive portion 300 preferably may not have any adhesive portion. The non-adhesive portion 300 is configured to sit directly over the wearer's nipple, which might be irritated by the adhesive.

Preferably, the nipple cover 100 and non-adhesive portion 300 comprise a soft rubber, gel, foam, or silicone so as to mimic the softness of a user's nipple and to provide comfort when worn. Preferably, the nipple cover 100 and/or non-adhesive portion 300 may be made of any flexible, soft, or elastic material that is designed to cover the user's nipple in a comfortable manner. Preferably, the remaining adhesive portion on the inner surface 200 is of sufficient surface area, as shown in FIG. 5, to secure the nipple cover to the breast of the wearer. The adhesive portion may typically be position just outside of the user's nipple, such as on or just outside of the user's areola.

Preferably, the adhesive on the inner surface 200 of the nipple cover 100 may be substantially water resistant, such that it may continue to stick, even when subjected to underlying sweat.

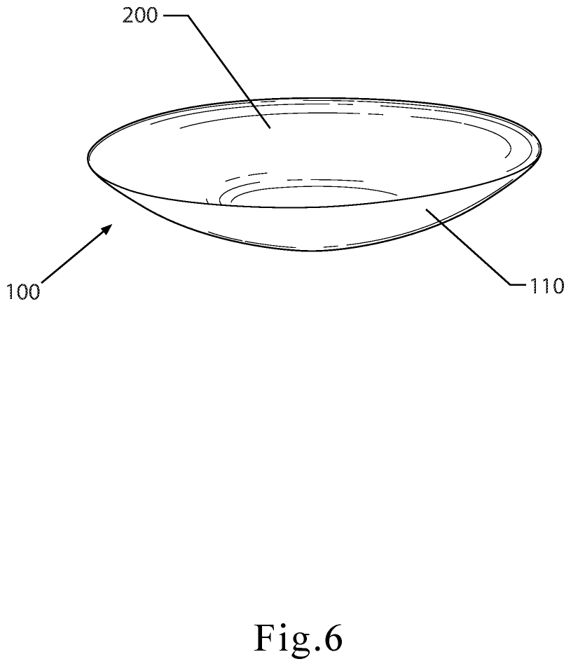

FIG. 6 is an illustration of an inner perspective view of one embodiment of a nipple cover. FIG. 6 shows that the nipple cover 100 may be concave on the inner surface 200 and convex on an outer surface 110. The inner surface 200 of the nipple cover 100 may be configured to form fittingly engage with the nipple and/breast area of a wearer, and the outer surface 110 may simulate a non-visible smooth tip of the wearer's breast. FIG. 6 also shows that the nipple cover 100 may have an interior, which is between the outer surface 110 and the inner surface 200, such that the nipple cover 100 has a thickness that is greater than the than the combined thickness of the inner surface 200 and the outer surface 110. FIG. 6 shows that the nipple cover 100 may be substantially lensed-shaped.

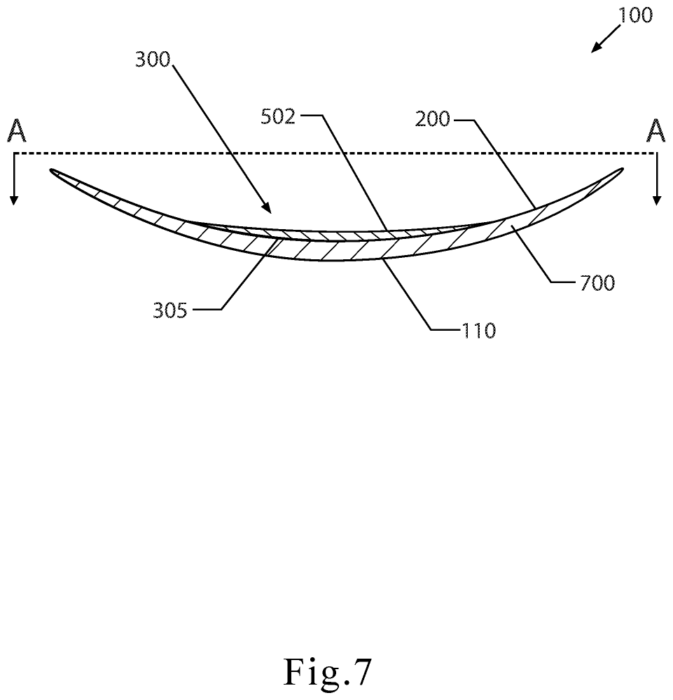

FIG. 7 is an illustration of a cross-section view of one embodiment of the nipple cover of FIG. 5. FIG. 7 shows that the nipple cover 100 may have an interior 700, an outer surface 110, an inner surface 200, a non-adhesive portion 300, which may have inner surface 502 and outer surface 305. FIG. 7 shows that the non-adhesive portion 300 matingly engages with the inner surface 110, such that the inner surface of the nipple cover has an adhesive portion and a non-adhesive portion, which is configured to directly cover the nipple.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit of the present disclosure. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A nipple cover, comprising:
an interior;
an outer surface; and
an inner surface, wherein said inner surface comprises an adhesive portion;
wherein said interior is between said outer surface and said inner surface;
wherein said outer surface is convex and said inner surface is concave, such that said nipple cover is curved and wherein said inner surface is configured to matingly engage with a wearer's breast, such that said wearer's nipple is covered by said nipple cover.

2. The nipple cover of claim 1, wherein said inner surface further comprises a non-adhesive portion.

3. The nipple cover of claim 2, wherein said non-adhesive portion is at a center of said inner surface, such that said non-adhesive portion is surrounded by said adhesive portion.

4. The nipple cover of claim 3, wherein said non-adhesive portion is separable from said nipple cover.

5. The nipple cover of claim 3, wherein said non-adhesive portion is unitary with said nipple cover.

6. The nipple cover of claim 3, wherein said nipple cover is made from a flexible and soft material.

7. The nipple cover of claim 6, wherein said nipple cover is made from one or more of the materials selected from the group of materials consisting of: natural rubber; synthetic rubber; silicone; silicone rubber; neoprene; foam rubber; nitrile; latex; and combinations thereof.

8. A nipple cover, comprising:
an interior;
an outer surface;
an inner surface, wherein said inner surface comprises an adhesive which entirely covers said inner surface; and
a non-adhesive portion;
wherein said interior is between said outer surface and said inner surface;
wherein said inner surface and said non-adhesive portion are configured to matingly engage, such that said non-adhesive portion is positioned at a center of said inner surface;
wherein said inner surface is concave; and
wherein said inner surface and said non-adhesive portion are configured to matingly engage with a wearer's breast, such that a nipple of said wearer is covered by said nipple cover.

9. The nipple cover of claim 8, wherein said non-adhesive portion directly covers said nipple, such that said adhesive does not engage directly with said nipple.

10. The nipple cover of claim 9, wherein said interior, said outer surface, said inner surface, and said non-adhesive portion are made from one or more of the materials selected from the group of materials consisting of: natural rubber; synthetic rubber; silicone; silicone rubber;
neoprene; foam rubber; nitrile; latex; and combinations thereof.

11. A nipple cover, comprising:
an outer surface;
an inner surface, wherein said inner surface comprises an adhesive which entirely covers said inner surface; and
a non-adhesive portion;
wherein said inner surface and said non-adhesive portion are configured to matingly engage, such that said non-adhesive portion is positioned at a center of said inner surface;
wherein said inner surface is concave;
wherein said inner surface and said non-adhesive portion are configured to matingly engage with a wearer's breast, such that a nipple of said wearer is covered by said nipple cover; and
wherein said non-adhesive portion directly covers said nipple, such that said adhesive does not engage directly with said nipple.

12. The nipple cover of claim 11, wherein said interior, said outer surface, said inner surface, and said non-adhesive portion are made from one or more of the materials selected from the group of materials consisting of: natural rubber; synthetic rubber; silicone; silicone rubber; neoprene; foam rubber; nitrile; latex; and combinations thereof.
